(19) United States
(12) Patent Application Publication
(10) Pub. No.: US 2004/0023979 A1
(43) Pub. Date: Feb. 5, 2004

(54) Process for making substituted thiazolyl-amino pyridines

(76) Inventors: Matthew M. Zhao, Edison, PA (US); Jingjun Yin, Green Brook, NJ (US)

Correspondence Address: MERCK AND CO INC, P O BOX 2000, RAHWAY, NJ, 070650907

(21) Appl. No.: 10/607,056

(22) Filed: Jun. 26, 2003

Related U.S. Application Data
(60) Provisional application No. 60/395,837, filed on Jul. 15, 2002.

(51) Int. Cl. A61K 31/496; C07D 417/14

(52) U.S. Cl. 514/253.1; 544/360

(57) ABSTRACT

The present invention relates to a process for preparing substituted thiazolyl-amino pyridines, which are capable of inhibiting, modulating and/or regulating signal transduction of both receptor-type and non-receptor type tyrosine kinases.

### BACKGROUND OF THE INVENTION

The present invention relates to a process for making substituted thiazolyl-amino pyridines, which inhibit, regulate and/or modulate tyrosine kinase signal transduction, and may be used to treat tyrosine kinase-dependent diseases and conditions, such as angiogenesis, cancer, tumor growth, atherosclerosis, age related macular degeneration, diabetic retinopathy, inflammatory diseases, and the like in mammals.

Tyrosine kinases are a class of enzymes that catalyze the transfer of the terminal phosphate of adenosine triphosphate to tyrosine residues in protein substrates. Tyrosine kinases play critical roles in signal transduction for a number of cell functions via substrate phosphorylation. Though the exact mechanisms of signal transduction is still unclear, tyrosine kinases have been shown to be important contributing factors in cell proliferation, carcinogenesis and cell differentiation.

Tyrosine kinases can be categorized as receptor type or non-receptor type. Receptor type tyrosine kinases have an extracellular, a transmembrane, and an intracellular portion, while non-receptor type tyrosine kinases are wholly intracellular.

The receptor-type tyrosine kinases are comprised of a large number of transmembrane receptors with diverse biological activity. In fact, about twenty different subfamilies of receptor-type tyrosine kinases have been identified. One tyrosine kinase subfamily, designated the HER subfamily, is comprised of EGFR, HER2, HER3, and HER4. Ligands of this subfamily of receptors include epithileal growth factor, TGF-α, amphiregulin, HB-EGF, betacellulin and heregulin. Another subfamily of these receptor-type tyrosine kinases is the insulin subfamily, which includes INS-R, IGF-IR, and IR-R. The PDGF subfamily includes the PDGF-α and β receptors, CSFIR, c-kit and FLK-II. Then there is the FLK family which is comprised of the kinase insert domain receptor (KDR), fetal liver kinase-1 (FLK-1), fetal liver kinase-4 (FLK-4) and the fms-like tyrosine kinase-1 (flt-1). The PDGF and FLK families are usually considered together due to the similarities of the two groups. For a detailed discussion of the receptor-type tyrosine kinases, see Plowman et al., DN&P 7(6):334-339, 1994, which is hereby incorporated by reference.

The non-receptor type of tyrosine kinases is also comprised of numerous subfamilies, including Src, Frk, Btk, Csk, Abl, Zap70, Fes/Fps, Fak, Jak, Ack, and LIMK. Each of these subfamilies is further sub-divided into varying receptors. For example, the Src subfamily is one of the largest and includes Src, Yes, Fyn, Lyn, Lck, Blk, Hck, Fgr, and Yrk. The Src subfamily of enzymes has been linked to oncogenesis. For a more detailed discussion of the non-receptor type of tyrosine kinases, see Bolen Oncogene, 8:2025-2031 (1993), which is hereby incorporated by reference.

Both receptor-type and non-receptor type tyrosine kinases are implicated in cellular signaling pathways leading to numerous pathogenic conditions, including cancer, psoriasis and hyperimmune responses.

Several receptor-type tyrosine kinases, and the growth factors that bind thereto, have been suggested to play a role in angiogenesis, although some may promote angiogenesis indirectly (Mustonen and Alitalo, J. Cell Biol. 129:895-898, 1995). One such receptor-type tyrosine kinase is fetal liver kinase 1 or FLK-1. The human analog of FLK-1 is the kinase insert domain-containing receptor KDR, which is also known as vascular endothelial cell growth factor receptor 2 or VEGFR-2, since it binds VEGF with high affinity. Finally, the murine version of this receptor has also been called NYK (Oelrichs et al., Oncogene 8(1):11-15, 1993). VEGF and KDR are a ligand-receptor pair that play an important role in the proliferation of vascular endothelial cells, and the formation and sprouting of blood vessels, termed vasculogenesis and angiogenesis, respectively.

Angiogenesis is characterized by excessive activity of vascular endothelial growth factor (VEGF). VEGF is actually comprised of a family of ligands (Klagsburn and D'Amore, Cytokine & Growth Factor Reviews 7:259-270, 1996). VEGF binds the high affinity membrane-spanning tyrosine kinase receptor KDR and the related fms-like tyrosine kinase-1, also known as Flt-1 or vascular endothelial cell growth factor receptor 1 (VEGFR-1). Cell culture and gene knockout experiments indicate that each receptor contributes to different aspects of angiogenesis. KDR mediates the mitogenic function of VEGF whereas Flt-1 appears to modulate non-mitogenic functions such as those associated with cellular adhesion. Inhibiting KDR thus modulates the level of mitogenic VEGF activity. In fact, tumor growth has been shown to be susceptible to the antiangiogenic effects of VEGF receptor antagonists. (Kim et al., Nature 362, pp. 841-844, 1993).

Solid tumors can therefore be treated by tyrosine kinase inhibitors since these tumors depend on angiogenesis for the formation of the blood vessels necessary to support their growth. These solid tumors include histiocytic lymphoma, cancers of the brain, genitourinary tract, lymphatic system, stomach, larynx and lung, including lung adenocarcinoma and small cell lung cancer. Additional examples include cancers in which overexpression or activation of Raf-activating oncogenes (e.g., K-ras, erb-B) is observed. Such cancers include pancreatic and breast carcinoma. Accordingly, inhibitors of these tyrosine kinases are useful for the prevention and treatment of proliferative diseases dependent on these enzymes.

The angiogenic activity of VEGF is not limited to tumors. VEGF accounts for most of the angiogenic activity produced in or near the retina in diabetic retinopathy. This vascular growth in the retina leads to visual degeneration culminating in blindness. Ocular VEGF mRNA and protein are elevated by conditions such as retinal vein occlusion in primates and decreased pO₂ levels in mice that lead to neovascularization. Intraocular injections of anti-VEGF monoclonal antibodies or VEGF receptor immunofusions inhibit ocular neovascularization in both primate and rodent models. Regardless of the cause of induction of VEGF in human diabetic retinopathy, inhibition of ocular VEGF is useful in treating the disease.

Expression of VEGF is also significantly increased in hypoxic regions of animal and human tumors adjacent to areas of necrosis. VEGF is also upregulated by the expression of the oncogenes ras, raf, src and mutant p53 (all of which are relevant to targeting cancer). Monoclonal anti-VEGF antibodies inhibit the growth of human tumors in nude mice. Although these same tumor cells continue to express VEGF in culture, the antibodies do not diminish their mitotic rate. Thus tumor-derived VEGF does not function as an autocrine mitogenic factor. Therefore, VEGF contributes to tumor growth in vivo by promoting angiogenesis through its paracrine vascular endothelial cell chemotactic and mitogenic activities. These monoclonal antibodies also inhibit the growth of typically less well vascularized human colon cancers in athymic mice and decrease the number of tumors arising from inoculated cells.

Viral expression of a VEGF-binding construct of Flk-1, Flt-1, the mouse KDR receptor homologue, truncated to eliminate the cytoplasmic tyrosine kinase domains but retaining a membrane anchor, virtually abolishes the growth of a transplantable glioblastoma in mice presumably by the dominant negative mechanism of heterodimer formation with membrane spanning endothelial cell VEGF receptors. Embryonic stem cells, which normally grow as solid tumors in nude mice, do not produce detectable tumors if both VEGF alleles are knocked out. Taken together, these data indicate the role of VEGF in the growth of solid tumors. Inhibition of KDR or Flt-1 is implicated in pathological angiogenesis, and these receptors are useful in the treatment of diseases in which angiogenesis is part of the overall pathology, e.g., inflammation, diabetic retinal vascularization, as well as various forms of cancer since tumor growth is known to be dependent on angiogenesis. (Weidner et al., N. Engl. J. Med., 324, pp. 1-8, 1991).

A number of compounds have been identified as inhibiting tyrosine kinase signal transduction, in particular as inhibitors of KDR. Several of these KDR inhibitors are characterized by a substituted thiazolyl-amino pyridinyl moiety, such as those illustrated in PCT Publication WO 01/17995.

Accordingly, a practical, efficient synthesis of substituted thiazolyl-amino pyridines is desirable and is an object of this invention.

### SUMMARY OF THE INVENTION

The present invention relates to a process for preparing substituted substituted thiazolyl-amino pyridines, such as those illustrated in Formula I

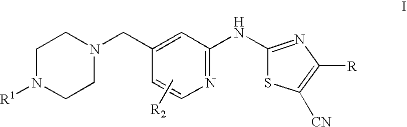

which are capable of inhibiting, modulating and/or regulating signal transduction of both receptor-type and non-receptor type tyrosine kinases.

### DETAILED DESCRIPTION OF THE INVENTION

A first embodiment of the instant invention is a process for preparing substituted thiazolyl-amino pyridines, such as those illustrated by Formula I:

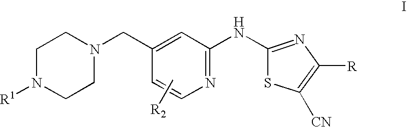

or a pharmaceutically acceptable salt or stereoisomer thereof, wherein

R is H, unsubstituted or substituted C₁-C₁₀ alkyl or unsubstituted or substituted aryl;

R¹ is —C(═O)NR³H;

R²is

1) H,

2) OH,

3) OC₁-C₆ alkyl,

4) C₁-C₆ alkyl, or

5) halo; and

R³ is C₁-C₆ alkyl;

which comprises the steps of:

a) preparing a slurry of a compound of Formula II

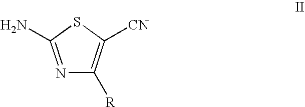

(where R is defined above), a compound of Formula III

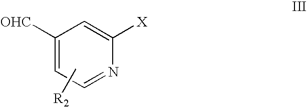

(where X is a halo and R² is defined above) and a base in a solvent;

b) adding a palladium catalyst and a bisphosphine ligand to the slurry to produce a coupling product of Formula IV

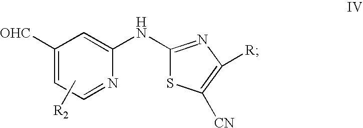

c) adding a piperazine-urea of Formula V

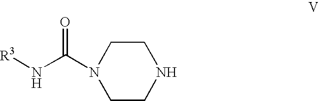

to the coupling product of Formula IV; and

d) completing a reductive amination to produce the compound of Formula I.

A further embodiment of the first embodiment is a process comprising the steps of:

a) preparing a slurry of a compound of Formula II

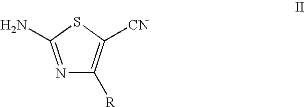

(where R is defined above), a compound of Formula III

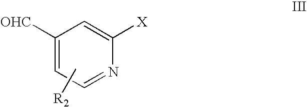

(where X is a halo and R² is defined above) and a phosphate in a solvent;

b) adding Pd₂(dba)₃ and Xantphos to the slurry to produce a coupling product of Formula IV

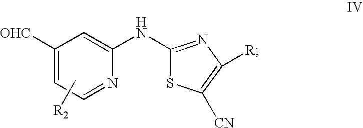

c) adding a piperazine-urea of Formula V

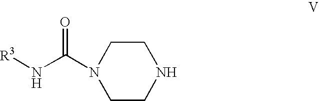

to the coupling product of Formula IV; and

d) completing a reductive amination to produce the compound of Formula I.

Another embodiment of the first embodiment for preparing a compound of Formula I comprises the steps of:

a) preparing a slurry of a compound of Formula II

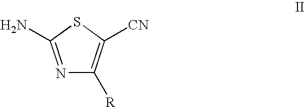

(where R is defined above), a compound of Formula III

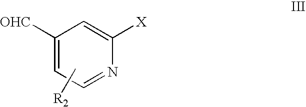

(where X is a halo and R² is defined above) and a carbonate in a solvent;

b) adding Pd₂(dba)₃ and Xantphos to the slurry to produce a coupling product of Formula IV

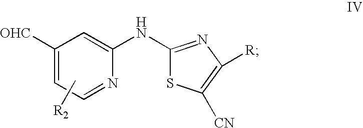

c) adding a piperazine-urea of Formula V

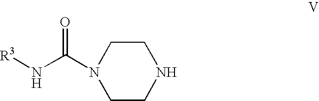

to the coupling product of Formula IV; and

d) completing a reductive amination to produce the compound of Formula I.

A second embodiment of the instant invention is a process for preparing 4-[2-(5-cyano-thiazol-2-ylamino)-pyridin-4-ylmethyl]-piperazine-1-carboxylic acid methylamide which comprises the steps of:

a) preparing a slurry of 2-chloro-4-formylpyridine, 2-aminothiazole and K₃PO₄ in toluene;

b) adding Pd₂(dba)₃ and Xantphos to the slurry to produce a coupling product;

c) adding N-methylaminocarbonylpiperazine in DMAc to the coupling product; and

d) completing a reductive amination by adding Et₃N, acetic acid and NaBH(OAc)₃ to produce 4-[2-(5-cyano-thiazol-2-ylamino)-pyridin-4-ylmethyl]-piperazine-1-carboxylic acid methylamide.

In a further embodiment of the second embodiment described above is the process which further comprises the step of adding Pd₂(dba)₃ and Xantphos to the slurry and heating to a temperature of about 60° C. to about 100° C. to produce a coupling product.

A third embodiment of the instant invention is the process for preparing a compound of Formula I which comprises the steps of:

a) preparing a slurry of a compound of Formula II

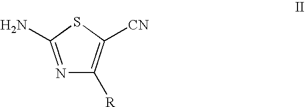

(where R is defined above), a compound of Formula III

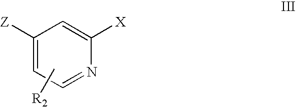

(where Z is CN or CO₂H; X is a halo and R² is defined above) and a base in a solvent;

b) adding a palladium catalyst and a bisphosphine ligand to the slurry to produce a coupling product of Formula IV

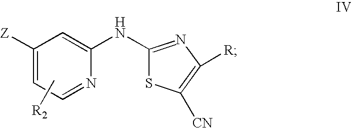

c) reducing the coupling product of Formula IV;

d) adding a piperazine-urea of Formula V

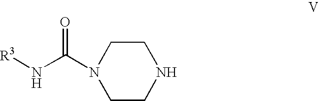

to the coupling product of Formula IV; and

e) completing a reductive amination to produce the compound of Formula I.

A fourth embodiment of the instant invention is the process for preparing a compound of Formula I which comprises the steps of:

a) preparing a slurry of a compound of Formula II

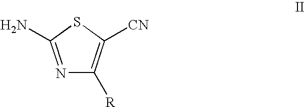

(where R is defined above), a compound of Formula III

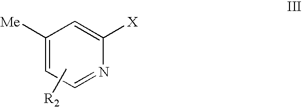

(where X is a halo and R² is defined above) and a base in a solvent;

b) adding a palladium catalyst and a bisphosphine ligand to the slurry to produce a coupling product of Formula IV

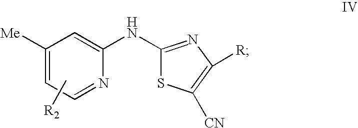

c) halogenating the coupling product of Formula IV;

d) adding a piperazine-urea of Formula V

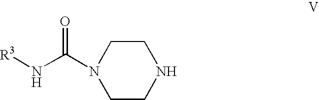

to the coupling product of Formula IV; and

e) completing a reductive amination to produce the compound of Formula I.

A fifth embodiment of the instant invention is the process for preparing a compound of Formula I which comprises the steps of:

a) preparing a slurry of a compound of Formula II

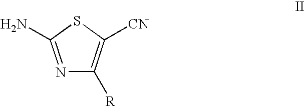

(where R is defined above), a compound of Formula III

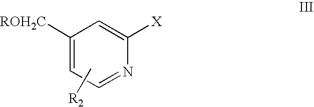

(where X is a halo and, R and R² are defined above) and a base in a solvent;

b) adding a palladium catalyst and a bisphosphine ligand to the slurry to produce a coupling product of Formula IV

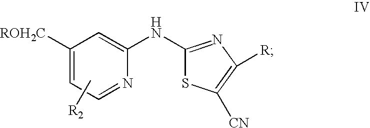

c) adding a piperazine-urea of Formula V

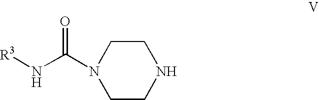

to the coupling product of Formula IV; and

d) completing a reductive amination to produce the compound of Formula I.

A sixth embodiment of the instant invention is the process for preparing Xantphos comprising the steps of:

a) adding MTBE, 9,9-dimethylxanthene and TMEDA to produce a solution;

b) adding s-BuLi to the solution to produce a mixture;

c) slowly adding Ph₂PCl to produce a resulting mixture;

d) aging the resulting mixture and adding more Ph₂PCl; and

e) filtering to isolate Xantphos.

These and other aspects of the invention will be apparent from the teachings contained herein.

“Tyrosine kinase-dependent diseases or conditions” refers to pathologic conditions that depend on the activity of one or more tyrosine kinases. Tyrosine kinases either directly or indirectly participate in the signal transduction pathways of a variety of cellular activities including proliferation, adhesion and migration, and differentiation. Diseases associated with tyrosine kinase activities include the proliferation of tumor cells, the pathologic neovascularization that supports solid tumor growth, ocular neovascularization (diabetic retinopathy, age-related macular degeneration, and the like) and inflammation (psoriasis, rheumatoid arthritis, and the like).

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted.

When any substituent and/or variable occurs more than one time in any constituent, its definition on each occurrence is independent at every other occurrence. Also, combinations of substituents and variables are permissible only if such combinations result in stable compounds. Lines drawn into the ring systems from substituents indicate that the indicated bond may be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” and “alkylene” are intended to include both branched and unbranched, cyclic and acyclic saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement and may be cyclic or acyclic. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, cyclopropyl, methyl-cyclopropyl, 2,2-dimethyl-cyclobutyl, 2-ethyl-cyclopentyl, cyclohexyl, and so on. In some instances, definitions may appear for the same variable reciting both alkyl and cycloalkyl when a different number of carbons is intended for the respective substituents. The use of both terms in one definition should not be interpreted to mean in another definition that “alkyl” does not encompass “cycloalkyl” when only “alkyl” is used.

“Alkoxy” represents an alkyl group of indicated number of carbon atoms as defined above attached through an oxygen bridge.

If no number of carbon atoms is specified, the term “alkenyl” refers to a non-aromatic hydrocarbon radical, which may be branched or unbranched and cyclic or acyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl, cyclohexenyl, methylenylcyclohexenyl, and so on.

The term “alkynyl” refers to a hydrocarbon radical, which may be branched or unbranched and cyclic or acyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃) CH₂CH(CH₃)Ph, and so on.

As used herein, “aryl” is intended to mean phenyl and substituted phenyl, including moieties with a fused benzo group. Examples of such aryl elements include phenyl, naphthyl, tetrahydronaphthyl, indanyl, biphenyl, phenanthryl, anthryl or acenaphthyl. In cases where the aryl substituent is bicyclic, it is understood that attachment is via the phenyl ring. Unless otherwise indicated, “aryl” includes phenyls substituted with one or more substituents.

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

As appreciated by those of skill in the art, “halo” or “halogen” as used herein is intended to include chloro, fluoro, bromo and iodo.

The term “heterocycle” or “heterocyclyl” as used herein is intended to mean a 5- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrathydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, aziridinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

The alkyl, alkylene, alkenyl, alkynyl, cycloalkyl, aryl, heteroaryl and heterocyclyl substituents may be unsubstituted or unsubstituted, unless specifically defined otherwise. For example, a (C₁-C₆)alkyl may be substituted with one, two or three substituents selected from F, Cl, Br, CF₃, N₃, NO₂, NH₂, oxo, —OH, —O(C₁-C₆ alkyl), S(O)₀₋₂, (C₁-C₆ alkyl) S(O)₀₋₂-, (C₁-C₆ alkyl)S(O)₀₋₂(C₁-C₆ alkyl)-, C₃-C₁₀ cycloalkyl, C₂-C₆ alkenyl, C₂-C₆ alkynyl, —C(O)NH, (C₁-C₆ alkyl) C(O)NH—, H₂NC(NH)—, (C₁-C₆ alkyl)C(O)—, —O(C₁-C₆ alkyl)CF₃, (C₁-C₆ alkyl)OC(O)—, (C₁-C₆ alkyl)O(C₁-C₆ alkyl)-, (C₁-C₆ alkyl)C(O)₂(C₁-C₆ alkyl)-, (C₁-C₆ alkyl)OC(O)NH—, aryl, benzyl, heterocycle, aralkyl, heterocyclylalkyl, halo-aryl, halo-benzyl, halo-heterocycle, cyano-aryl, cyano-benzyl and cyano-heterocycle. In this case, if one substituent is oxo and the other is OH, the following are included in the definition: —(C═O)CH₂CH(OH)CH₃, —(C═O)OH, —CH₂(OH)CH₂CH(O), and so on.

In an embodiment of the instant process, compounds of Formulae II and III are added, with a base to a solvent. Preferably, the base is a carbonate or phosphate. A palladium catalyst and a bisphosphine ligand are added to the slurry to produce a coupling product of Formula IV. In a preferred embodiment of the instant invention, the process comprises adding Pd₂(dba)₃ and Xantphos to the slurry. In a more preferred embodiment, the process further comprises adding Pd₂(dba)₃ and Xantphos to the slurry and heating to a temperature of about 60° C. to about 100° C. to produce a coupling product. A piperazine-urea of Formula V is added to the coupling product. Then reductive amination is done to produce a compound of Formula I.

The base compound utilized in the instant invention includes, but is not limited to, phosphates, bicarbonates, carbonates, alkoxides or hydroxides. Preferably, the base is a phosphate or carbonate. Examples of phosphates that may be used in the instant process may include, but are not limited to, cesium phosphate, lithium phosphate, potassium phosphate, sodium phosphate, and the like. Examples of carbonates that be may utilized may include, but are not limited to, cesium carbonate, lithium carbonate, potassium carbonate, sodium carbonate, and the like.

As used herein, a “solvent” may include, but is not limited to, water, alcohols, unchlorinated or chlorinated hydrocarbons, nitriles, ketones, ethers, polar aprotic solvents or mixtures thereof. Types of alcohols that may be used include, but are not limited to, methanol, ethanol, n-propanol, i-propanol, butanol or an alkoxyethanol. Types of unchlorinated hydrocarbons include, but are not limited to, toluene or xylene. Types of chlorinated hydrocarbons include, but are not limited to, dichloro-methane, chloroform, chlorobenzene or ODCB. Types of nitriles include, but are limited to, acetonitrile, propionitrile, benzonitrile or tolunitrile. Types of ketones include, but are not limited to, acetone, MEK, MIBK and cyclohexanone. Types of ethers include, but are not limited to, diethyl ether, MTBE, THF, DME and DEM. Types of polar aprotic solvents include, but are not limited to, formamide, DMF, DMA, NMP, DMPU, DMSO, and sulfolane. Preferably, the solvent is DMF, DMAc, Toluene, Acetonitirile, or an ether. Most preferably, the solvent is DMF or DMAc.

Examples of palladium catalysts that may be used in the instant invention include, but are not limited to, Pd₂(dba)₃, Pd(dba)₂, Pd(OAc)₂; Pd(PPh₃)₄, PdCl₂, PdBr₂, PdF₂, PdI₂ and the like. More preferably, the palladium catalyst is Pd₂(dba)₃ or Pd(dba)₂.

Types of bisphosphine ligands that may be utilized in the instant invention include, but are not limited to, Xantphos, BINAP, DPPF, DPPP, DPEPhos, and the like. Most preferably, Xantphos is used.

In embodiments of the instant invention, the process comprises the step of reducing the coupling product of Formula IV. This reduction may be performed using standard techniques, such as those described in Smith, M. B., March, J.; Advanced Organic Chemistry; Reactions, Mechanisms, and Structures., 5th ed., John Wiley & Sons, New York, 2001.

In the fourth embodiment of the instant invention, the process comprises the step of halogenating the coupling product of Formula IV. As used herein, “halogenating” may be done by the addition of a halogenating agent in order to attach a halo or halogen to a compound. Halogenating agents may include, but are not limited to Br₂, NBS, 1,3-dibromo-5,5-dimethylhydantoin, pyr.HBr₃, NCS, Cl₂, 1,3-dichloro-5,5-dimethylhydantoin, pyr.HCl₃, F₂, 1,3-difluro-5,5-dimethylhydantoin and the like, to a solution or mixture. Most preferably, the instant process comprises the step of brominating the coupling product of Formula IV by adding a “brominating agent”, such as Br₂, NBS, 1,3-dibromo-5,5-dimethylhydantoin, pyr.HBr₃, and the like.

The salts of the compounds prepared by the instant processes include the conventional salts of the compounds, e.g., inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, trifluoroacetic and the like.

With respect to compounds which contain an acid moiety, a salt may take the form, for example, —COOM, where M is a negative charge, which is balanced by a counterion, e.g., an alkali metal cation such as sodium or potassium. Other pharmaceutically acceptable counterions may be calcium, magnesium, zinc, ammonium, or alkylammonium cations such as tetramethylammonium, tetrabutylammonium, choline, triethylhydroammonium, meglumine, triethanolhydroammonium and the like.

Some of the abbreviations that may be used in the description of the chemistry and in the Examples include:

| | |
|---|---|
| ACN | Acetonitrile; |
| Ac₂O | Acetic anhydride; |
| AcOH | Acetic acid; |
| AIBN | 2,2′-Azobisisobutyronitrile; |
| BINAP | 2,2′-Bis(diphenylphosphino)-1,1′ binaphthyl; |
| Bn | Benzyl; |
| BOC/Boc | tert-Butoxycarbonyl; |
| BSA | Bovine Serum Albumin; |
| CAN | Ceric Ammonia Nitrate; |
| CBz | Carbobenzyloxy; |
| CI | Chemical Ionization; |
| DBA | dibenzanthracene; |
| DBAD | Di-tert-butyl azodicarboxylate; |
| DBU | 1,8-Diazabicyclo[5.4.0]undec-7-ene; |
| DCE | 1,2-Dichloroethane; |
| DEAD | diethylazodicarboxylate; |
| DEM | diethoxymethane; |
| DIAD | diisopropylazodicarboxylate; |
| DIEA | N,N-Diisopropylethylamine; |
| DMAC | N,N-dimethylacetamide; |
| DMAP | 4-Dimethylaminopyridine; |
| DME | 1,2-Dimethoxyethane; |
| DMF | N,N-Dimethylformamide; |
| DMPU | 1,3-Dimethyl-3,4,5,6-tetrahydro-2-(1H)-pyrimidinone; |
| DMSO | Methyl sulfoxide; |
| DPAD | dipiperidineazodicarbonyl; |
| DPEPhos | 1,1′-(Bisdiphenylphosphino)diphenylether; |
| DPPA | Diphenyiphosphoryl azide; |
| DPPF | 1,1′-(Bisdiphenylphosphino)ferrocene; |
| DPPP | 1,3-(Bisdiphenylphosphino)propane; |
| DTT | Dithiothreitol; |
| EDC | 1-(3-Dimethylaminopropyl)-3-ethyl- |
| | carbodiimide-hydrochloride; |
| EDTA | Ethylenediaminetetraacetic acid; |
| ES | Electrospray; |
| ESI | Electrospray ionization; |
| Et₂O | Diethyl ether; |
| Et₃N | Triethylamine; |
| EtOAc | Ethyl acetate; |
| EtOH | Ethanol; |
| FAB | Fast atom bombardment; |
| HEPES | 4-(2-Hydroxyethyl)-1-piperazineethanesulfonic acid; |
| HOAc | Acetic acid; |
| HMTA | Hexamethylenetetramine; |
| HOBT | 1-Hydroxybenzotriazole hydrate; |
| HOOBT | 3-Hydroxy-1,2,2-benzotriazin-4(3H)-one; |
| HPLC | High-performance liquid chromatography; |
| HRMS | High Resolution Mass Spectroscopy; |
| KOtBu | Potassium tert-butoxide; |
| LAH | Lithium aluminum hydride; |
| LCMS | Liquid Chromatography Mass Spectroscopy; |
| MCPBA | m-Chloroperoxybenzoic acid; |
| Me | Methyl; |
| MEK | Methyl ethyl ketone; |
| MeOH | Methanol; |
| MIBK | Methyl isobutyl ketone; |
| Ms | Methanesulfonyl; |
| MS | Mass Spectroscopy; |
| MsCl | Methanesulfonyl chloride; |
| MsOH | methanesulfonic acid; |
| MTBE | tert-butyl methyl ether; |
| n-Bu | n-butyl; |
| n-Bu₃P | Tri-n-butylphosphine; |
| NaHMDS | Sodium bis(trimethylsilyl)amide; |
| NBS | N-Bromosuccinimide; |
| NMP | N-Methyl pyrrolidinone; |
| ODCB | Ortho Dichlorobenzene, or 1,2-dichlorobenzene; |
| Pd(PPh₃)₄ | Palladium tetrakis(triphenylphosphine); |
| Pd₂(dba)₂ | Tris(dibenzylideneacetone)dipalladium (0) |
| Ph | phenyl; |
| PMSF | α-Toluenesulfonyl fluoride; |
| Py or pyr | Pyridine; |
| PYBOP | Benzotnazol-1-yloxytripyrrolidinophosphonium |
| (or PyBOP) | hexafluorophosphate; |
| RPLC | Reverse Phase Liquid Chromatography; |
| rt (or RT) | Room Temperature; |
| t-Bu | tert-Butyl; |
| TBAF | Tetrabutylammonium fluoride; |
| TBSCl | tert-Butyldimethylsilyl chloride; |
| TFA | Trifluoroacetic acid; |
| THF | Tetrahydrofuran; |
| TIPS | Triisopropylsilyl; |
| TMEDA | N,N,N′,N′-Tetramethylethylenediamine; |
| TMS | Tetramethylsilane; |
| Tr | Trityl; |
| TsOH | P-Toluenesulfonic acid; |
| Xantphos | 9,9-dimethyl-4,5-bis(diphenylphosphino)xanthene. |

The use of the process of the instant invention to prepare KDR inhibitors (such as those described in PCT Publ. WO 01/17995) is illustrated in the following schemes, in addition to other standard manipulations that are known in the literature or exemplified in the experimental procedures. These schemes and examples, therefore, are not limited by the compounds listed or by any particular substituents employed for illustrative purposes.

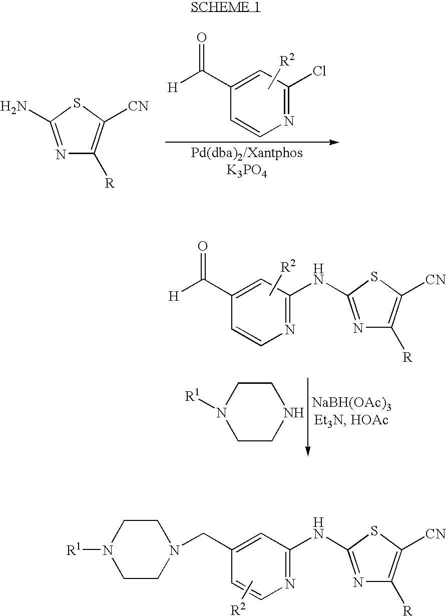

### EXAMPLES

Examples provided are intended to assist in a further understanding of the invention. Particular materials employed, species and conditions are intended to be further illustrative of the invention and not limiting of the reasonable scope thereof.

### Example 1

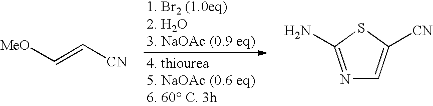

Bromine (2.88 Kg, 18.0 mole) is added to a solution of 3-methoxyacrylonitrile (1.50 Kg, 18.0 mole, mixture of cis-/trans-isomers) in acetonitrile (3.00 L) at 5-10° C. The mixture is aged for 20 minutes, then pre-cooled water (˜5° C., 12.0 L) is added and vigorous stirred for 1 hour.

NaOAc.3H₂O, (2.21 Kg, 16.2 mole, 0.90 equiv.) is added and stirred for 15 minutes and then thiourea (1.51 Kg, 19.80 mole, 1.10 equiv.) is added (endothermic dissolution followed by ˜10-15° C. exotherm in ˜0.5 h). The mixture is aged at 15° C. for 1.5 hour, then more NaOAc.3H₂O (1.47 Kg, 0.60 equiv.) is added. It is slowly heated to 60° C. in 1 hour and aged for 3 hours at 60° C. then cooled to 10° C.

NaOH (10 N, 1.13 L, 0.625 equiv.) is added to adjust the pH to 3.8-4.0. After aging for 1 hour, the product is filtered and washed with water (11.5 L). Drying give 1.86 Kg of the crude aminothiazole as a brown solid, (97A %), 80.7% yield.

The crude product is dissolved into acetone (35 L) at 50° C. and treated with Darco KB-B (380 g) for 2 hours. It is filtered through a Solka-Floc pad and then rinsed with acetone (5 L). The filtrate is concentrated in vacuo to ˜7 L (˜5 L residue acetone). Heptane (10 L) is added in 0.5 hour and the slurry is aged for 1 hour. The product is filtered and the filter cake is washed with 2/1 heptane/acetone (6 L). Drying at rt affords 1.72 Kg of the aminothiazole as a pinkish solid, 75% yield. HPLC conditions: Ace-C8 4.6×250 mm column; linear gradient: 5-80% MeCN in 12 minutes, 0.1% H₃PO₄ in the aqueous mobile phase; Flow rate: 1.50 ml/min; UV detection at 220 nm.

### Example 2

### Preparation of 9,9-dimethyl-4,5-bis(diphenylphosphino)xanthene, Xantphos

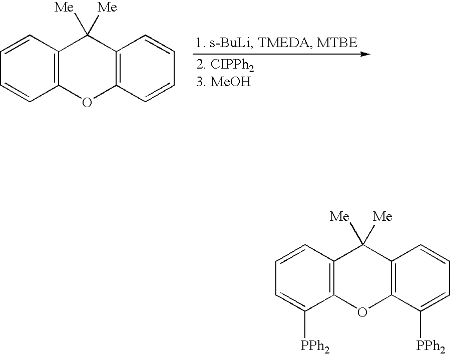

To a 1L round bottom flask (RBF) are added MTBE (500 mL), 9,9-dimethylxanthene (26.65 g) and TMEDA (30.6 g). After degassing the solution, s-BuLi (155 g, 1.3 M in cyclohexane) is cannulated into a dropping funnel and then slowly added over 30 min while maintaining the batch temperature at 10-20° C. The mixture is then aged for 16 h at room temperature. Ph₂PCl is added slowly via a dropping funnel while maintain the mildly exothermic reaction at 10-20° C.

˜60% of the Ph₂PCl (30 mL) is added in 0.5 hour. The mixture is aged for 15 minutes before addition of the remaining Ph₂PCl. After aged for 5.5 h at room temperature, the reaction is quenched with MeOH (2.0 mL). The product is filtered and the slightly yellow solid is washed consecutively with MeOH (200 mL), water (200 mL), MeOH (200 mL) and MTBE (200 mL) and dried to give an off-white solid as product (54.8 g, 77% yield).

### Example 2A

### Preparation of 9,9-dimethyl-4,5-bis(diphenylphosphino)xanthene, Xantphos

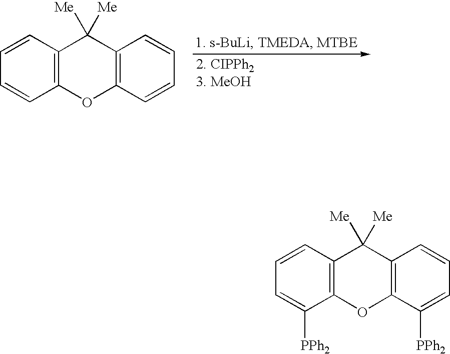

To a 5L round bottom flask (RBF) are added MTBE (2.5 L), 9,9-dimethylxanthene (131.4 g, 0.60 mole) and TMEDA (155 g, 1.32 mole). After degassing the solution, s-BuLi (1.11 L, 1.3 M in cyclohexane, 1.44 mole) is cannulated into a dropping funnel and then slowly added over 60 min while maintaining the batch temperature at 6-10° C. The mixture is then aged for 14 h at room temperature. Ph₂PCl is added slowly via a dropping funnel while maintain the mildly exothermic reaction at 10-20° C.

˜60% of the Ph₂PCl (175 mL, 0.93 mole) is added in 0.5 hour. The mixture is aged for 10 minutes before addition of the remaining Ph₂PCl (120 mL, 0.63 mole). After aged for 5 h at room temperature, the reaction is quenched with MeOH (9.9 mL, 0.24 mole). The product is filtered and the slightly yellow solid is washed consecutively with MTBE (250 mL), MeOH (2×250 mL), water (2×300 mL), MeOH (2×250 mL) and MTBE (250 mL) and dried to give an off-white solid as product (304.2 g, 88% yield).

### Example 3

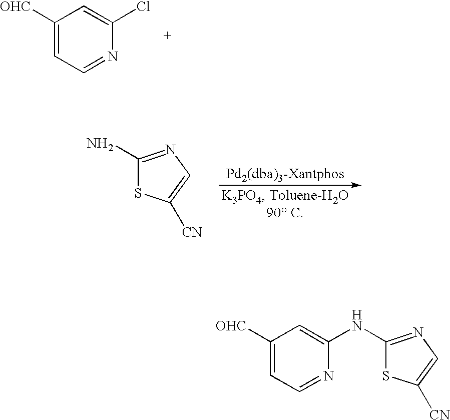

A slurry of 2-chloro-4-formylpyridine (1.49 Kg, 10.5 mole, 1.05 equiv), 2-aminothiazole (1.27 Kg, 10.0 mole, 1.0 equiv), K₃PO₄ (2.34 Kg, 11.0 mole, 1.1 equiv) in toluene (20 L) is degassed by two vacuum/nitrogen cycles. Pd₂(dba)₃ (114.5 g, 0.125 mmol, 2.5 mol % Pd) and Xantphos (159 g, 0.275 mole, 2.75 mol %) are then added and the mixture is degassed by one vacuum/nitrogen cycle followed by bubbling nitrogen through the slurry for 10 minutes. The mixture is heated to 60° C. and degassed water (90 mL, 5.0 mole, 0.5 equiv) was added over 5 minutes. The mixture is then heated to 90° C. and aged for 8 h.

It is cooled to room temperature and filtered. The filter cake is washed with toluene (20 L) until very little DBA is observed in the wash. DMAc (24 L) is added to the filter cake to dissolve the product. The insoluble is filtered off and washed with more DMAc (6 L). The filtrate is acidified with concentrate HCl (110 mL) to pH 2.7. Water (3 L) is added and the mixture is concentrated at 40-50° C. under vacuum to remove most of the residual toluene by azeotropic distillation. More water (3×1L) is added as the distillation progress.

The mixture is seeded and then water (13 L) is added at a rate of ˜1.3 L/h. The product is filtered and washed with 5/4 DMAc/water (4.0 L×2), water (4.0 L), acetone (4 L×2), and then oven dried at 40C. under vacuum (100 mmHg) with nitrogen sweep to give 1.92 Kg product (94.5 wt %, 97A %).

### Example 4

### Preparation of N-benzyl-N′-methylaminocarbonylpiperazine Dihydrate

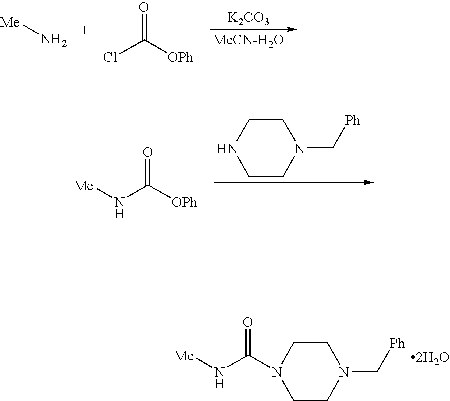

To a 50-L 3-neck RBF is added H₂O (6.0 L) followed by K₂CO₃ (4.56 Kg) with stirring. It is cooled to 10° C. Acetonitrile (12 L) and methylamine (40 wt % in water, 1.40 Kg) are added and the mixture is cooled to 0-5° C. Phenyl chloroformate (2.59 Kg) is then added as quickly as possible while maintaining the exothermic reaction at <15° C. 1-Benzylpiperazine is added 15 min after addition of phenyl chloroformate and the biphasic mixture is heated to 70° C. After aging for 1 h at 70° C., the reaction mixture was concentrated under vacuum to remove most of the MeCN.

NaOH (7.5L 5 N NaOH) is added and the mixture is seeded. The suspension is then cooled to rt and aged for 1 hour. The product is filtered and the filter cake is washed with cold NaOH (0.5 N aq, 4 L×2) and then ice-cold water (4 L×2). It is purified by recrystallization from toluene (15 L) to remove any dibenzylpiperazine impurity. NaOH is used to remove phenol. The solubility of the product in water is somewhat high (7 mg/mL) at rt, so iced water is used for the wash.

### Example 5

### Preparation of N-methylaminocarbonylpiperazine Hydrochloride (2)

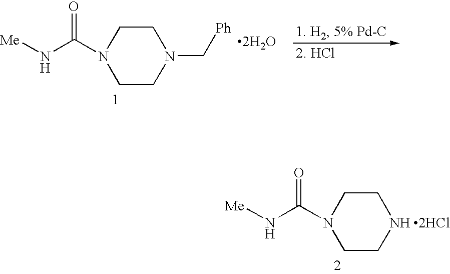

HCl (74 mL 12 N, 0.10 eq) is added to MeOH (7 L) and then piperazine urea 1 (2.69 Kg, 10.0 mol) is added. The mixture is hydrogenated using 5% Pd/C (180 g) under 40 psi of hydrogen pressure at 40° C. for 18 h. Pd/C is slurried in MeOH (1 L) and transferred by vacuum.

After confirming the completion of the reaction, the mixture is filtered through a pad of Solka-Floc and washed with MeOH (2 L) then IPA (4 L). The colorless solution is concentrated to ˜5-6 L at ca 40° C. under vacuum. IPA (5 L) is added followed by HCl (12 N aq, 0.767 L, 0.92 eq) until the pH of the solution becomes ˜3. The mixture is then concentrated under vacuum and flushed with more IPA (5+5 L) to a final volume of 6 L. KF of the supernatant should be <1 w % water. It is then aged at 15° C. for 5 h.

The resulting white crystals are filtered and washed with IPA (4 L). It is then dried in a vacuum oven at 40° C. with slow nitrogen sweep to give 1.53 Kg of 2 (99 w %, 95% corrected yield).

### Example 6

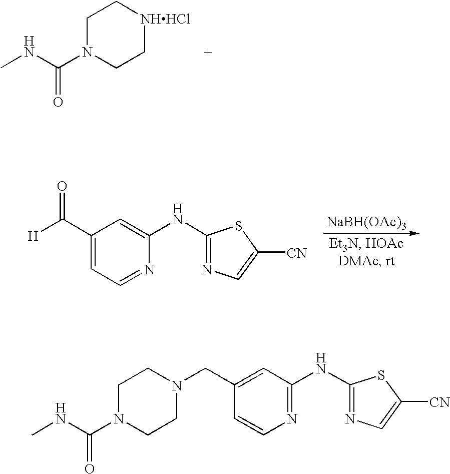

To a slurry of the pyridine aldehyde (2.19 Kg, 94.5 w %, 9.00 mole) and the piperazine urea HCl salt (1.79 Kg, 9.90 mmol) in DMAc (13.5 L) is added Et₃N (1.00 Kg, 9.90 mole) followed by acetic acid (2.16 Kg, 36.0 mole) with cooling (15° C.). After aging for 0.5 h, NaBH(OAc)₃ (2.29 Kg, 10.8 mole) is added in 8 portions (25 minutes/portion).

The mixture is stirred for 1 hour and the completion of the reaction confirmed by HPLC. Water (6.8 L) is added slowly (14 h) to complete the crystallization. Seed with monohydrate of the free base after ˜1-2 L of water has been added.

The product is filtered after aging for 3 hours and the filter cake washed with 3/2 DMAc/water (6.7 L), then 1/1 acetone/water (6 L) then acetone (2×4 L). Oven drying at 40 C. with slow nitrogen sweep afforded 2.71 Kg of the crude product. HPLC assay, 95.4 w %, 98.9A %, 80.4% correct yield. KF=2.5 w %.

### Assays

The compounds prepared utilizing the instant invention described in the Examples were tested by the assays described below and were found to have kinase inhibitory activity. Other assays are known in the literature and could be readily performed by those of skill in the art (see, for example, Dhanabal et al., Cancer Res. 59:189-197; Xin et al., J. Biol. Chem. 274:9116-9121; Sheu et al., Anticancer Res. 18:4435-4441; Ausprunk et al., Dev. Biol. 38:237-248; Gimbrone et al., J. Natl. Cancer Inst. 52:413-427; Nicosia et al., In Vitro 18:538-549).

I. VEGF Receptor Kinase Assay

VEGF receptor kinase activity is measured by incorporation of radio-labeled phosphate into polyglutamic acid, tyrosine, 4:1 (pEY) substrate. The phosphorylated pEY product is trapped onto a filter membrane and the incorporation of radio-labeled phosphate quantified by scintillation counting.

Materials

VEGF Receptor Kinase

The intracellular tyrosine kinase domains of human KDR (Terman, B. I. et al. Oncogene (1991) vol. 6, pp. 1677-1683.) and Flt-1 (Shibuya, M. et al. Oncogene (1990) vol. 5, pp. 519-524) were cloned as glutathione S-transferase (GST) gene fusion proteins. This was accomplished by cloning the cytoplasmic domain of the KDR kinase as an in frame fusion at the carboxy terminus of the GST gene. Soluble recombinant GST-kinase domain fusion proteins were expressed in Spodoptera frugiperda (Sf21) insect cells (Invitrogen) using a baculovirus expression vector (pAcG2T, Pharmingen).

The other materials used and their compositions were as follows:

Lysis buffer: 50 mM Tris pH 7.4, 0.5 M NaCl, 5 mM DTT, 1 mM EDTA, 0.5% triton X-100, 10% glycerol, 10 mg/mL of each leupeptin, pepstatin and aprotinin and 1 mM phenylmethylsulfonyl fluoride (all Sigma).

Wash buffer: 50 mM Tris pH 7.4, 0.5 M NaCl, 5 mM DTT, 1 mM EDTA, 0.05% triton X-100, 10% glycerol, 10 mg/mL of each leupeptin, pepstatin and aprotinin and 1 mM phenylmethylsulfonyl fluoride.

Dialysis buffer: 50 mM Tris pH 7.4, 0.5 M NaCl, 5 mM DTT, 1 mM EDTA, 0.05% triton X-100, 50% glycerol, 10 mg/mL of each leupeptin, pepstatin and aprotinin and 1 mM phenylmethylsuflonyl fluoride.

10× reaction buffer: 200 mM Tris, pH 7.4, 1.0 M NaCl, 50 mM MnCl₂, 10 mM DTT and 5 mg/mL bovine serum albumin (Sigma).

Enzyme dilution buffer: 50 mM Tris, pH 7.4, 0.1 M NaCl, 1 mM DTT, 10% glycerol, 100 mg/mL BSA.

10× Substrate: 750 μg/mL poly (glutamic acid, tyrosine; 4:1) (Sigma).

Stop solution: 30% trichloroacetic acid, 0.2 M sodium pyrophosphate (both Fisher).

Wash solution: 15% trichloroacetic acid, 0.2 M sodium pyrophosphate.

Filter plates: Millipore #MAFC NOB, GF/C glass fiber 96 well plate.

Method

A. Protein Purification

1. Sf21 cells were infected with recombinant virus at a multiplicity of infection of 5 virus particles/cell and grown at 27° C. for 48 hours.

2. All steps were performed at 4° C. Infected cells were harvested by centrifugation at 1000× g and lysed at 4° C. for 30 minutes with 1/10 volume of lysis buffer followed by centrifugation at 100,000×g for 1 hour. The supernatant was then passed over a glutathione Sepharose column (Pharmacia) equilibrated in lysis buffer and washed with 5 volumes of the same buffer followed by 5 volumes of wash buffer. Recombinant GST-KDR protein was eluted with wash buffer/10 mM reduced glutathione (Sigma) and dialyzed against dialysis buffer.

B. VEGF Receptor Kinase Assay

1) Add 5 μl of inhibitor or control to the assay in 50% DMSO.

2) Add 35 μl of reaction mix containing 5 μl of 10× reaction buffer, 5 μl 25 mM ATP/10 μCi [³³P]ATP (Amersham), and 5 μl 10× substrate.

3) Start the reaction by the addition of 10 μl of KDR (25 nM) in enzyme dilution buffer.

4) Mix and incubate at room temperature for 15 minutes.

5) Stop by the addition of 50 μl stop solution.

6) Incubate for 15 minutes at 4° C.

7) Transfer a 90 μl aliquot to filter plate.

8) Aspirate and wash 3 times with wash solution.

9) Add 30 μl of scintillation cocktail, seal plate and count in a Wallac Microbeta scintillation counter.

II. Human Umbilical Vein Endothelial Cell Mitogenesis Assay

Human umbilical vein endothelial cells (HUVECs) in culture proliferate in response to VEGF treatment and can be used as an assay system to quantify the effects of KDR kinase inhibitors on VEGF stimulation. In the assay described, quiescent HUVEC monolayers are treated with vehicle or test compound 2 hours prior to addition of VEGF or basic fibroblast growth factor (bFGF). The mitogenic response to VEGF or bFGF is determined by measuring the incorporation of [³H] thymidine into cellular DNA.

Materials

HUVECs: HUVECs frozen as primary culture isolates are obtained from Clonetics Corp. Cells are maintained in Endothelial Growth Medium (EGM; Clonetics) and are used for mitogenic assays described in passages 3-7 below.

Culture Plates: NUNCLON 96-well polystyrene tissue culture plates (NUNC #167008).

Assay Medium: Dulbecco's modification of Eagle's medium containing 1 g/mL glucose (low-glucose DMEM; Mediatech) plus 10% (v/v) fetal bovine serum (Clonetics).

Test Compounds: Working stocks of test compounds are diluted serially in 100% dimethylsulfoxide (DMSO) to 400-fold greater than their desired final concentrations. Final dilutions to 1× concentration are made directly into Assay Medium immediately prior to addition to cells.

10× Growth Factors: Solutions of human VEGF₁₆₅ (500 ng/mL; R&D Systems) and bFGF (10 ng/mL; R&D Systems) are prepared in Assay Medium.

10× [³H]Thymidine: [Methyl-³H]thymidine (20 Ci/mmol; Dupont-NEN) is diluted to 80 μCi/mL in low-glucose DMEM.

Cell Wash Medium: Hank's balanced salt solution (Mediatech) containing 1 mg/mL bovine serum albumin (Boehringer-Mannheim).

Cell Lysis Solution: 1 N NaOH, 2% (w/v) Na₂CO₃.

Method

1. HUVEC monolayers maintained in EGM are harvested by trypsinization and plated at a density of 4000 cells per 100 μL Assay Medium per well in 96-well plates. Cells are growth-arrested for 24 hours at 37° C. in a humidified atmosphere containing 5% CO₂.

2. Growth-arrest medium is replaced by 100 μL Assay Medium containing either vehicle (0.25% [v/v] DMSO) or the desired final concentration of test compound. All determinations are performed in triplicate. Cells are then incubated at 37° C. with 5% CO₂ for 2 hours to allow test compounds to enter cells.

3. After the 2-hour pretreatment period, cells are stimulated by addition of 10 μL/well of either Assay Medium, 10× VEGF solution or 10× bFGF solution. Cells are then incubated at 37° C. and 5% CO₂.

4. After 24 hours in the presence of growth factors, 10× [³H]thymidine (10 μL/well) is added.

5. Three days after addition of [³H]thymidine, medium is removed by aspiration, and cells are washed twice with Cell Wash Medium (400 μL/well followed by 200 μL/well). The washed, adherent cells are then solubilized by addition of Cell Lysis Solution (100 μL/well) and warming to 37° C. for 30 minutes. Cell lysates are transferred to 7-mL glass scintillation vials containing 150 μL of water. Scintillation cocktail (5 mL/vial) is added, and cell-associated radioactivity is determined by liquid scintillation spectroscopy.

Based upon the foregoing assays the comopunds of Formula I are inhibitors of VEGF and thus are useful for the inhibition of angiogenesis, such as in the treatment of ocular disease, e.g., diabetic retinopathy and in the treatment of cancers, e.g., solid tumors. The instant compounds inhibit VEGF-stimulated mitogenesis of human vascular endothelial cells in culture with IC₅₀ values between 0.01-5.0 μM. These compounds may also show selectivity over related tyrosine kinases (e.g., FGFR1 and the Src family; for relationship between Src kinases and VEGFR kinases, see Eliceiri et al., Molecular Cell, Vol. 4, pp. 915-924, December 1999).

III. FLT-1 Kinase Assay

Flt-1 was expressed as a GST fusion to the Flt-1 kinase domain and was expressed in baculovirus/insect cells. The following protocol was employed to assay compounds for Flt-1 kinase inhibitory activity:

1) Inhibitors were diluted to account for the final dilution in the assay, 1:20.

2) The appropriate amount of reaction mix was prepared at room temperature:

10× Buffer (20 mM Tris pH 7.4/0.1 M NaCl/1 mM DTT final)

0.1M MnCl₂ (5 mM final)

pEY substrate (75 μg/mL)

ATP/[³³P]ATP (2.5 μM/1 μCi final)

BSA (500 μg/mL final).

3) 5 μL of the diluted inhibitor was added to the reaction mix. (Final volume of 5 μL in 50% DMSO). To the positive control wells, blank DMSO (50%) was added.

4) 35 μL of the reaction mix was added to each well of a 96 well plate.

5) Enzyme was diluted into enzyme dilution buffer (kept at 4° C.).

6) 10 μL of the diluted enzyme was added to each well and mix (5 nM final). To the negative control wells, 10 μL 0.5 M EDTA was added per well instead (final 100 mM).

7) Incubation was then carried out at room temperature for 30 minutes.

8) Stopped by the addition of an equal volume (50 μL) of 30% TCA/0.1M Na pyrophosphate.

9) Incubation was then carried out for 15 minutes to allow precipitation.

10) Transfered to Millipore filter plate.

11) Washed 3× with 15% TCA/0.1M Na pyrophosphate (125 μL per wash).

12) Allowed to dry under vacuum for 2-3 minutes.

13) Dryed in hood for ˜20 minutes.

14) Assembled Wallac Millipore adapter and added 50 μL of scintillant to each well and counted.

IV. FLT-3 Kinase Assay

Flt-3 was expressed as a GST fusion to the Flt-3 kinase domain, and was expressed in baculovirus/insect cells. The following protocol was employed to assay compounds for Flt-3 kinase inhibitory activity:

1) Dilute inhibitors (account for the final dilution into the assay, 1:20)

2) Prepare the appropriate amount of reaction mix at room temperature.

10× Buffer (20 mM Tris pH 7.4/0.1 M NaCl/1 mM DTT final)

0.1M MnCl₂ (5 mM final)

pEY substrate (75 μg/mL)

ATP/[³³P]ATP (0.5 μM/L μCi final)

BSA (500 μg/mL final)

3) Add 5 μL of the diluted inhibitor to the reaction mix. (Final volume of 5 μL in 50% DMSO). Positive control wells—add blank DMSO (50%).

4) Add 35 μL of the reaction mix to each well of a 96 well plate.

5) Dilute enzyme into enzyme dilution buffer (keep at 4° C.).

6) Add 10 μL of the diluted enzyme to each well and mix (5-10 nM final). Negative control wells—add 10 μL 0.5 M EDTA per well instead (final 100 mM)

7) Incubate at room temperature for 60 minutes.

8) Stop by the addition of an equal volume (50 μL) of 30% TCA/0.1M Na pyrophosphate.

9) Incubate for 15 minutes to allow precipitation.

10) Transfer to Millipore filter plate.

11) Wash 3× with 15% TCA/0.1M Na pyrophosphate (125 μL per wash).

12) Allow to dry under vacuum for 2-3 minutes.

13) Dry in hood for ˜20 minutes.

14) Assemble Wallac Millipore adapter and add 50 μL of scintillant to each well and count.

What is claimed is:

1. A process for preparing a compound of Formula I: 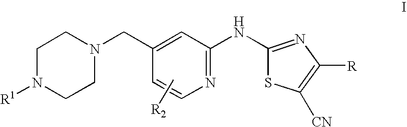
or a pharmaceutically acceptable salt or stereoisomer thereof, wherein
R is H, unsubstituted or substituted C₁-C₁₀ alkyl or unsubstituted or substituted aryl;
R¹ is —C(═O)NR³H;
R² is
1) H,
2) OH,
3) OC₁-C₆ alkyl,
4) C₁-C₆ alkyl, or
5) halo; and
R³ is C₁-C₆ alkyl;
which comprises the steps of:
a) preparing a slurry of a compound of Formula II 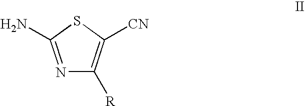
(where R is defined above), a compound of Formula III 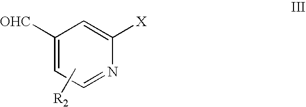
(where X is a halo and R² is defined above) and a base in a solvent;
b) adding a palladium catalyst and a bisphosphine ligand to the slurry to produce a coupling product of Formula IV 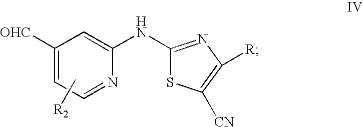
c) adding a piperazine-urea of Formula V 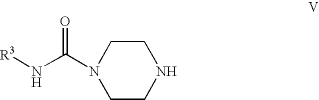
to the coupling product of Formula IV; and
d) completing a reductive amination to produce the compound of Formula I.

2. The process according to claim 1 comprising the steps of:
a) preparing a slurry of a compound of Formula II 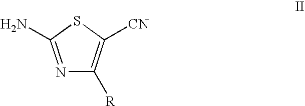
(where R is defined above), a compound of Formula III 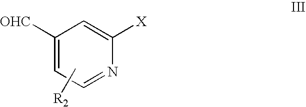
(where X is a halo and R² is defined above) and a phosphate in a solvent;
b) adding Pd₂(dba)₃ and Xantphos to the slurry to produce a coupling product of Formula IV 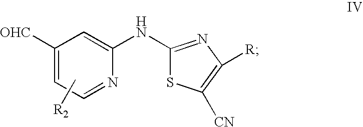
c) adding a piperazine-urea of Formula V 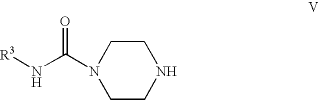
to the coupling product of Formula IV; and
d) completing a reductive amination to produce the compound of Formula I.

3. The process according to claim 1 which comprises the steps of:
a) preparing a slurry of a compound of Formula II 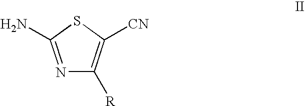
(where R is defined above), a compound of Formula III 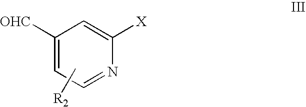
(where X is a halo and R² is defined above) and a carbonate in a solvent;
b) adding Pd₂(dba)₃ and Xantphos to the slurry to produce a coupling product of Formula IV 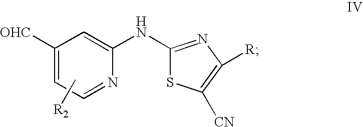
c) adding a piperazine-urea of Formula V 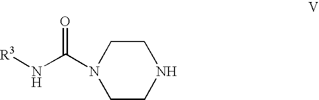
to the coupling product of Formula IV; and
d) completing a reductive amination to produce the compound of Formula I.

4. A process for preparing 4-[2-(5-cyano-thiazol-2-ylamino)-pyridin-4-ylmethyl]-piperazine-1-carboxylic acid methylamide which comprises the steps of:
a) preparing a slurry of 2-chloro-4-formylpyridine, 2-aminothiazole and K₃PO₄ in toluene;
b) adding Pd₂(dba)₃ and Xantphos to the slurry to produce a coupling product;
c) adding N-methylaminocarbonylpiperazine in DMAc to the coupling product; and
d) completing a reductive amination by adding Et₃N, acetic acid and NaBH(OAc)₃ to produce 4-[2-(5-cyano-thiazol-2-ylamino)-pyridin-4-ylmethyl]-piperazine-1-carboxylic acid methylamide.

5. The process according to claim 4 which further comprises the step of adding Pd₂(dba)₃ and Xantphos to the slurry and heating to a temperature of about 60° C. to about 100° C. to produce a coupling product.

6. A process for preparing a compound of Formula I 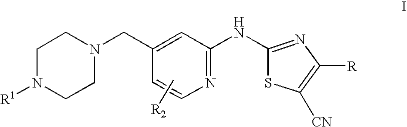
or a pharmaceutically acceptable salt or stereoisomer thereof, wherein
R is H, unsubstituted or substituted C₁-C₁₀ alkyl or unsubstituted or substituted aryl;
R¹ is —C(═O)NR³H;
R² is
1) H,
2) OH,
3) OC₁-C₆ alkyl,
4) C₁-C₆ alkyl, or
5) halo; and
R³ is C₁-C₆ alkyl;
which comprises the steps of:
a) preparing a slurry of a compound of Formula II 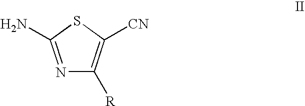
(where R is defined above), a compound of Formula III 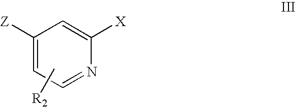
(where Z is CN or CO₂H; X is a halo and R² is defined above) and a base in a solvent;
b) adding a palladium catalyst and a bisphosphine ligand to the slurry to produce a coupling product of Formula IV 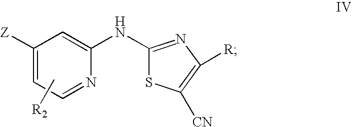
c) reducing the coupling product of Formula IV;
d) adding a piperazine-urea of Formula V 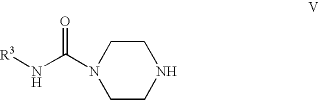
to the coupling product of Formula IV; and
e) completing a reductive amination to produce the compound of Formula I.

7. A process for preparing a compound of Formula I 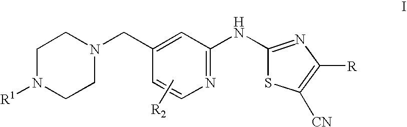
or a pharmaceutically acceptable salt or stereoisomer thereof, wherein
R is H, unsubstituted or substituted C₁-C₁₀ alkyl or unsubstituted or substituted aryl;
R¹ is —C(═O)NR³H;
R² is
1) H,
2) OH,
3) OC₁-C₆ alkyl,
4) C₁-C₆ alkyl, or
5) halo; and
R³ is C₁-C₆ alkyl;
which comprises the steps of:
a) preparing a slurry of a compound of Formula II 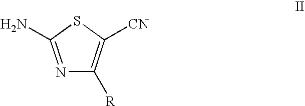
(where R is defined above), a compound of Formula III 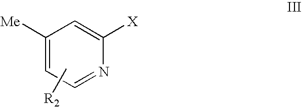
(where X is a halo and R² is defined above) and a base in a solvent;
b) adding a palladium catalyst and a bisphosphine ligand to the slurry to produce a coupling product of Formula IV 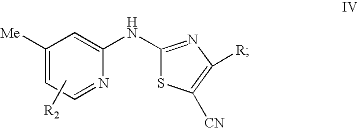
c) halogenating the coupling product of Formula IV;
d) adding a piperazine-urea of Formula V 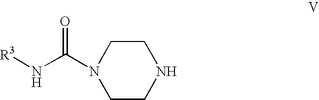
to the coupling product of Formula IV; and
e) completing a reductive amination to produce the compound of Formula I.

8. A process for preparing a compound of Formula I 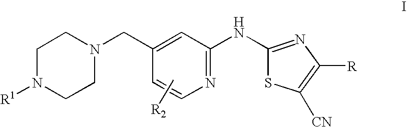
or a pharmaceutically acceptable salt or stereoisomer thereof, wherein
R is H, unsubstituted or substituted C₁-C₁₀ alkyl or unsubstituted or substituted aryl;
R¹ is —C(═O)NR³H;
R² is
1) H,
2) OH,
3) OC₁-C₆ alkyl,
4) C₁-C₆ alkyl, or
5) halo; and
R³ is C₁-C₆ alkyl;
which comprises the steps of:
a) preparing a slurry of a compound of Formula II 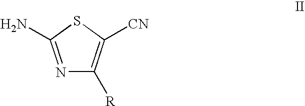
(where R is defined above), a compound of Formula III 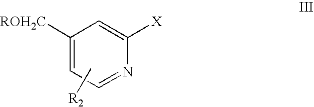
(where X is a halo and, R and R² are defined above) and a base in a solvent;
b) adding a palladium catalyst and a bisphosphine ligand to the slurry to produce a coupling product of Formula IV 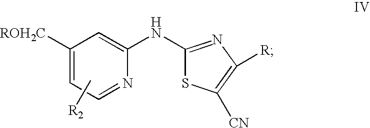
c) adding a piperazine-urea of Formula V 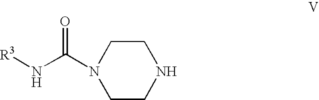
to the coupling product of Formula IV; and
d) completing a reductive amination to produce the compound of Formula I.

9. A process for preparing Xantphos comprising the steps of:
a) adding MTBE, 9,9-dimethylxanthene and TMEDA to produce a solution;
b) adding s-BuLi to the solution to produce a mixture;
c) slowly adding Ph₂PCl to produce a resulting mixture;
d) aging the resulting mixture and adding more Ph₂PCl; and
e) filtering to isolate Xantphos.
